(19) United States
(12) Patent Application Publication
(10) Pub. No.: US 2013/0330827 A1
(43) Pub. Date: Dec. 12, 2013

(54) In Vitro Methodology for Predicting in Vivo Absorption Time of Bioabsorbable Polymeric Implants and Devices

(71) Applicant: Ethicon, Inc., (US)

(72) Inventors: Dennis D. Jamiolkowski, Long Valley, NJ (US); Benjamin D. Fitz, Port Murray, NJ (US); Dachuan Yang, Hillsborough, NJ (US)

(73) Assignee: ETHICON, INC., Somerville, NJ (US)

(21) Appl. No.: 13/682,275

(22) Filed: Nov. 20, 2012

Related U.S. Application Data
(60) Provisional application No. 61/565,856, filed on Dec. 1, 2011.

(51) Int. Cl. G01N 33/44

(52) U.S. Cl. CPC G01N 33/442
USPC 436/34

(57) ABSTRACT

A novel in vitro methodology for predicting the in vivo behavior, such as absorption time or mechanical strength retention, of biodegradable polymeric implants and medical devices. The present invention provides a novel in vitro methodology, hydrolysis profiling, for studying the degradation of absorbable polymers. Accuracy and reproducibility have been established for selected test conditions. Data from this in vitro method are correlated to in vivo absorption data, allowing for the prediction of accurate in vivo behaviors, such as absorption times.

### CROSS REFERENCE TO RELATED APPLICATION

This application claims priority to provisional application No. 61/565,856, filed Dec. 1, 2011.

### TECHNICAL FIELD

The field of art to which this patent application relates is methods for predicting the in vivo absorption time of bioabsorbable polymeric implants and medical devices, more specifically, in vitro test methods for predicting in vivo absorption times of bioabsorbable polymeric implants and medical devices in humans and mammals.

### BACKGROUND OF THE INVENTION

Bioabsorbable polymers are known to have great utility in the medical field. They are particularly useful as surgical implants and medical devices. The bioabsorbable polymeric materials are designed to provide adequate strength and retention of mechanical properties in vivo to accomplish the function of the implant or medical device during the healing process, while degrading at a controlled and desired rate so that the device is essentially eliminated from the patient's body after natural healing has occurred and the implant or device is no longer required. Surgical implants and medical devices made from bioabsorbable polymers often provide a superior patient outcome.

Synthetic absorbable polymers are an important class of materials used in a variety of implantable medical devices. Many of these devices, such as surgical sutures and surgical meshes, are used for soft tissue wound closure applications. There are also orthopedic applications of such polymers for hard tissue (e.g., bone), including fixation devices such as pins, screws, plates, suture anchors, and longer lasting suture materials.

Medical devices prepared from synthetic absorbable polymers may be classified as filamentous or non-filamentous products. Filamentous products include suture materials (both in monofilament form as well as multifilament form) and mesh products (based on knitted, woven, and nonwoven architectures). Table 1 (contained herein below) lists some of the various fiber-based products that are derived from synthetic absorbable polymers. These fibers are generally made by conventional melt extrusion and orientation processes.

The other class, non-filamentous products, are frequently fabricated by injection molding. Table 2 (contained herein below) lists many of these types of non-filamentous devices. They include suture anchors, bone pins and plates, ligating clips, and rivets. In addition to devices that find value by exhibiting high mechanical properties, there are applications in which utility is based on diffusion characteristics such as carriers and protective layers used in controlled drug delivery applications, often as coatings, microspheres or microcapsules.

The global market for medical devices based upon this class of polymers is immense and continues playing an ever-expanding role with exciting new applications on the horizon, addressing unmet patient needs. These new applications may include their use as scaffolds for cell transplantation and tissue engineering in regenerative medicine. Existing materials may not satisfy all the challenges that lie ahead in this field. Material scientists continue to work to increase the performance characteristics of these bioabsorbable materials and devices, looking to provide better mechanical properties such as better strength and/or stiffness, or allowing these mechanical properties to be retained longer and last longer in vivo.

It is important to be able to predict the in vivo absorption times of biodegradable polymeric implants and medical devices for a number of reasons. There must be a degree of correlation between the length of time that the implants can retain their strength and mechanical properties in vivo and the length of time for the healing process to progress to the point that the tissue can resume its normal functioning. Premature absorption and loss of mechanical strength and other mechanical properties may lead to a catastrophic failure resulting in injury to the patient or a life threatening event requiring immediate medical intervention. In addition, it is beneficial to design the implant or device to have the minimum mass necessary to function adequately during the healing process.

As new absorbable polymers are being developed for medical devices and implants, a key issue is the length of time it will take for the material to disappear in the body, i.e., to absorb. Related to this issue is the desire to engineer medical devices and implants from bioabsorbable polymers that have desired absorption profiles in vivo. The definitive answer to this question is usually provided by preclinical studies using radiolabeled materials following the absorption, distribution, metabolism, and excretion of these materials and degradation products. The hydrolysis by-products may be converted to CO₂ and exhaled or may be excreted in urine or feces. Radio-labeled materials can also be used to determine the fate or disposition of the materials, i.e., to determine whether the by-products are actually excreted or sequestered in target organs. Other important means for studying bioabsorption include histology in which a measurement of the cross-sectional area of the implant is made as a function of time. Of course, histology also provides important information on the tissue reaction that the implant elicits.

Traditional in vivo methods of assessing bioabsorption rates are expensive, time consuming, and obviously require the use of laboratory animals. Preclinical testing may be adequate to obtain regulatory approval and demonstrate safety and efficacy; however, there may be instances where human clinical trials may be required. In the case of the radiolabeled studies, typically an appropriately labeled C₁₄ monomer must be synthesized and scrupulously purified. The monomer must then be safely polymerized, and the resulting radioactive polymer must be converted to a test article possessing appropriate mechanical properties. In the case of a suture, this will typically require a strong, properly oriented fiber.

Broadly, from a humanitarian aspect, in vitro testing is preferred over animal testing, provided that as useful, valid data is generated. Additionally, although in vitro testing data can be collected under simulated physiological conditions, it is also desirable to collect such data in an accelerated fashion. Testing can be accelerated in some cases by changing temperature, pH, other parameters, or combinations thereof to obtain data in a quicker fashion than real-time testing. Product development cycle time can potentially be shortened by getting an early indication of performance, whether the focus is on the polymer composition or processing conditions used to make the article.

Clearly, it would be advantageous to be able to estimate the rate of breakdown of a new bioabsorbable material, whether it is a different chemistry or an altered polymer morphology, without having to resort to radio-labeled or histological studies. It is known that the biodegradation of absorbable polyesters used in medical devices occurs via hydrolysis of ester linkages, with the by-product being acid generation. Generation of acid groups may not be troublesome to the surrounding tissue if the body's biological mechanisms can appropriately neutralize them as they are created. However, if a material undergoes too rapid hydrolysis, the tissues at the implant site may not be able to maintain a proper pH, thus causing undue inflammation [1].

As just pointed out, chemistry and polymer morphology affect device performance characteristics. Important, clinically significant characteristics include dimensional stability, mechanical properties, rate of loss of mechanical properties post-implantation, and rate of absorption. Chemistry plays a dominant role in determining hydrolysis rates; the hydrolysis rate then greatly influences the tissue absorption profile and biological compatibility.

But chemistry is not the only factor that influences performance. Samples of the same polymer, indistinguishable in all chemical features, with the same molecular weight distributions, can behave very differently with regard to their biological and mechanical performance if they exhibit different polymer morphologies. Polymer morphology refers to the shape or pattern in assemblies of the macromolecular chains; at its very simplest, it can refer to crystallinity level. However, in addition to the relative amount of the crystalline and amorphous phases, morphological characterization of a semi-crystalline polymer includes the amount of molecular orientation present (both crystalline and amorphous), the nature of the crystal structure, and the size distribution of the crystals. These characteristics are usually influenced by the thermal and mechanical or stress history that the polymer was exposed to during processing and device fabrication.

It can be appreciated that the relationships are complex: chemistry and processing affect morphology; chemistry and morphology affect hydrolysis rates; and, hydrolysis rates affect biological performance. It is thus vital to fully characterize the absorbable medical device or implant with regard to composition and morphology, and to understand the impact of these factors on in vivo absorption time.

Over the years, various conventional techniques have been employed to follow the degradation of absorbable polyesters. Some in vivo studies examined loss of mechanical properties with time post-implantation. Of particular relevance to suture materials have been loss of breaking strength with time studies; these are often referred to as BSR (Breaking Strength Retention) studies. Other than BSR in vivo studies, real time (as well as accelerated) in vitro tests have also been described and are known. These methods, however, do not generally predict in vivo absorption time. To address absorption issues using in vitro methodologies, researchers have conducted mass loss studies. The deficiency in that approach is reduced accuracy when the material loses mechanical integrity and begins to disintegrate into smaller and smaller particles, leading to filtration and weight assessment challenges. Other methods that have been employed include following changes in molecular weight as a function of time [2]. This approach, however, is cumbersome to conduct on a routine basis. Sawhney and Hubble [3] have reported a method specific to lactic acid soluble degradants.

It is well known that one might follow ester hydrolysis of organic compounds by titration in an aqueous media [4-11]. Titration has also been used to provide information on the hydrolysis of a number of polyphosphates [12]. Tunc and co-workers [13] have described the use of accelerated in vitro pH-stat titration to estimate in vivo absorption times of alpha-hydroxyester polymers. Their methodology, however, did not compare in vitro testing results to in vivo absorption on a wide-range of absorbable materials; they only studied polymers and copolymers of lactide and glycolide. Polymers and copolymers of lactide and glycolide, in the absence of a plasticizer (including residual monomer) have glass transition temperatures between about 40° C. and 65° C., well above body temperature. The authors limited their test method to temperatures below the glass transition temperature of the polymers they studied; this low test temperature restriction severely limits their ability to collect data in an accelerated fashion. To compensate, it appears that Tunc and coworkers have utilized a linear extrapolation from early hydrolysis times to reduce testing duration. Collecting data only at an early hydrolysis stage may not be appropriate for absorbable materials having complex morphology if it is desired to predict total absorption time. Another area of concern with regard to using only data collected at early hydrolysis times is when the test article comprises a polymer of complex sequence distribution. Consider, for instance, an A-B block copolymer of 80/20 (mol %) epsilon-caprolactone and glycolide; in this case all of the caprolactone sequences are linked and the glycolide sequences are linked. Estimating the absorption time via the Tunc method would significantly underestimate the amount of time necessary to undergo complete in vivo absorption. This is because the glycolide sequences would hydrolyze well before the epsilon-caprolactone sequences, leaving a relatively intact poly(epsilon-caprolactone) mass.

Limiting the testing conditions to temperatures below the glass transition (Tg) of the polymers would be problematic for absorbable polymers with low Tg's, such as poly(p-dioxanone). Since all monofilament sutures have glass transition temperatures below room temperature, this important product class could not be tested by Tunc's method in view of his restriction.

Another known titration technique has been used to study the enzymatic degradation of poly(hydroxybutyrates) [14], as well as for the study of hydrolysis of short-chain polyesters [15].

Although conventional in vitro test methods are used to roughly predict in vivo bioabsorption behavior, there are deficiencies associated with their use. With some present methods, the data cannot be collected in an accelerated fashion. This is particularly troublesome for polymers having long absorption times. An example of this class of materials are those based on polymerimized lactide; corresponding devices are often used in the field of orthopedics. Having a means of obtaining estimates of absorption time in an accelerated manner speeds development time and helps in product optimization. Clearly in vitro testing is advantageous over in vivo testing from a humane aspect in that animal use is significantly reduced or even eliminated. The costs associated with in vivo testing are significantly higher than the costs associated with in vitro testing. As pointed out earlier, existing in vitro testing methods are fraught with experimental challenges and poor accuracy.

Accordingly there is a need in this art for novel methods of in vitro testing of bioabsorbable implants and medical devices that quickly, humanely, economically, accurately, and reproducibly predict in vivo bioabsorption times.

### SUMMARY OF THE INVENTION

A novel in vitro methodology for predicting the in vivo absorption time of bioabsorbable polymeric implants and medical devices is disclosed. The method provides for predicting the in vivo absorption time of synthetic absorbable polymers, their implants or medical devices formed therefrom, possessing hydrolysable linkages within the polymer chain, based on an in vitro test. The method has the following steps:
(a) subjecting a known quantity of test article of known in vivo absorption time to hydrolysis at a substantially constant pH and at a substantially constant test temperature above or at body temperature using a known concentration of titrating base, and recording the volume of titrating base with time;
(b) recording the time necessary to achieve a constant level of percent hydrolysis of the test article wherein said percent hydrolysis is 70 percent or greater;
(c) repeating steps (a) and (b) utilizing the test conditions selected for steps (a) and (b) with at least one different test article of different known in vivo absorption times;
(d) constructing an in vivo-in vitro correlation curve of in vivo absorption time versus in vitro hydrolysis time as recorded in step (b);
(e) subjecting a known quantity of test article of unknown in vivo absorption time to hydrolysis at the test conditions selected for steps (a) and (b) using a known concentration of titrating base, and recording the volume of titrating base with time; and,
(f) predicting the in vivo absorption time utilizing the correlation curve of step (d) and the in vitro hydrolysis time of step (e).

Yet another aspect of the present invention is a novel in vitro methodology for predicting the in vivo absorption time of bioabsorbable polymeric implants and medical devices. The method provides for predicting the in vivo absorption time of synthetic absorbable polymers, their implants or medical devices formed therefrom, possessing hydrolysable linkages within the polymer chain, based on an in vitro test. The method has the following steps:
(a) subjecting a known quantity of test article of known in vivo absorption time to hydrolysis at a substantially constant pH and at a substantially constant test temperature above or at body temperature using a known concentration of titrating base, and recording the volume of titrating base with time;
(b) recording the time necessary to achieve a constant level of percent hydrolysis of the test article wherein said percent hydrolysis is 70 percent or greater;
(c) constructing an in vivo-in vitro correlation curve of in vivo absorption time versus in vitro hydrolysis time as recorded in step (b);
(d) subjecting a known quantity of test article of unknown in vivo absorption time to hydrolysis at the test conditions selected for steps (a) and (b) using a known concentration of titrating base, and, recording the volume of titrating base with time; and,
(e) predicting the in vivo absorption time utilizing the correlation curve of step (c) and the in vitro hydrolysis time of step (d);

These and other aspects and advantages of the present invention will become more apparent from the following description and accompanying drawings:

### BRIEF DESCRIPTION OF THE DRAWINGS

FIG. 1 is a graph of precision and accuracy of hydrolysis profiler performance: six repetitions of hydrolysis of 80 mg glycolide monomer. Experimental conditions: pH 7.27, 75 mL of water, 0.05N NaOH, and 75° C. Plot of titration time-course or “hydrolysis profile”.

FIG. 2 illustrates hydrolysis profiles at pH 7.27 of 100 mg glycolide in 75 mL of water with 0.05N NaOH at selected temperatures.

FIG. 3 is a graph of hydrolysis kinetics of glycolide at pH 7.27 at selected temperatures.

FIG. 4 is an Arrhenius plot of rate constant of hydrolysis of linear dimers of glycolic acid.

FIG. 5 illustrates hydrolysis profiles of glycolide and lactide monomers at pH 7.27 at 75° C.

FIG. 6 is a graph illustrating temperature dependence of the hydrolysis half-time of VICRYL™ and VICRYL RAPIDE™ brand sutures.

FIG. 7 illustrates hydrolysis profiles of selected ETHICON brand sutures (100 mg of each suture).

FIG. 8 illustrates a correlation between in vivo and in vitro absorption times for selected ETHICON brand sutures.

FIG. 9 illustrates the dependence of suture hydrolysis time at 75° C. on fiber diameter of MONOCRYL brand monofilament suture.

FIG. 10 is a graph of Suture Breaking Strength Retention (BSR) as a function of extent of carboxylic acid group generation.

### DETAILED DESCRIPTION OF THE INVENTION

It should be noted that the terms absorbable and bioabsorbable when referring to synthetic polymers are used interchangeably herein. The hydrolysis profile method records as a function of time the amount of base needed to maintain the aqueous media at a selected constant pH while ester hydrolysis takes place. In doing so, it can be used to determine the time for achieving a relative fraction of hydrolysis, including complete hydrolysis. Those skilled in the art will recognize that conventional equipment may be used to conduct the method of the present invention. Equipment may include for example a pH probe, glass vessels with temperature control, automatic dosing systems, data recording and remote instrument control capability, etc., and equivalents thereof. Control, data collection and analysis and presentation may be via conventional and/or customized computers and conventional and/or customized software and equivalents thereof.

The method consists of hydrolytically degrading a test specimen while maintaining a constant pH. This is done by titrating with a standard base and measuring the quantity of base used as a function of time. The measurement and titration are conveniently automated.

As part of the novel method of the present invention, in vitro work is conducted to completely hydrolyze an absorbable polyester surgical implant device, such as sutures at constant pH and elevated temperature. It should also be recognized that complete hydrolysis is not always needed, but hydrolysis levels greater than about 90% are preferred. This may be accomplished using a conventional multi-neck round-bottomed flask equipped with a pH probe, temperature controller, and a controlled means of introducing a dilute sodium hydroxide solution through Teflon® tubing. An absorbable polyester surgical suture (or other absorbable test article) is added to this reactor containing, initially, only distilled water. The data can be recorded manually or with computer aid. In a preferred embodiment, the setup includes an electronic controller that takes the signal from the pH meter and causes a Teflon®-lined valve in the Teflon® tubing line to be opened in order to titrate the reaction so as to remain at a constant pre-determined pH set-point. Acid groups are generated as hydrolysis of the absorbable polyester suture (or bioabsorbable polymer test article) occurs, incrementally lowering the pH, as detected by the pH probe. The controller would then open the Teflon®-lined, electronically controlled valve, introducing base to titrate the mixture returning it to the pH set-point. The container of the dilute sodium hydroxide solution is mounted on an electronic balance so as to allow monitoring of the loss in weight as the NaOH solution is consumed during the hydrolysis process. Thus, through observation and manual recording, one can follow the extent of hydrolysis with time. With the use of computer control this basic methodology has been enhanced for convenience, accuracy, and standardization. It will be appreciated by those skilled in the art that the procedure can be performed manually without automatic controllers if desired, although not preferred.

The methodologies of the present invention may be applied to polymers possessing esters in their backbones. The methods may also be applicable, in modified form, to gain insight into the degradation of candidate polymer systems, for instance those containing esters in pendant groups. The pendant ester hydrolysis may lead to chain segment solubilization or in other instances, depending on the chemistry, lead to main chain degradation because of local pH changes, a so called “neighboring group effect”.

The hydrolysis profiler method presented here applies to conventional synthetic absorbable polyesters, polyanhydrides, and other polymers with hydrolytically degradable linkages, and equivalents thereof that yield acidic degradation products.

The bioabsorbable polymers that can be used to make devices that can be tested according to the method of the present invention include conventional biocompatible, bioabsorbable polymers including polymers selected from the group consisting of aliphatic polyesters, poly(amino acids), copoly(ether-esters), polyalkylene oxalates, polyalkylene diglycolates, polyamides, tyrosine-derived polycarbonates, poly(iminocarbonates), polyorthoesters, polyoxaesters, polyamidoesters, polyoxaesters containing amine groups, poly(anhydrides), polyphosphazenes, polypropylene fumarates), absorbable poly(ester urethanes), and combinations and blends thereof, and equivalents. The polyoxaesters include the polymers based on 3,6-dioxaoctanedioic acid, 3,6,9-trioxaundecanedioic acid, and the diacid known as polyglycol diacid, which can be made from the oxidation of low molecular weight polyethylene glycol.

Suitable polymers can be homopolymers or copolymers (random, block, segmented, tapered blocks, graft, triblock, etc.) having a linear, branched or star structure. Suitable monomers for making suitable polymers may comprise one or more of the following monomers: lactic acid (including L-lactic acid and D-lactic acid), lactide (including L-, D-, meso and D,L-mixtures), glycolic acid, glycolide, ε-caprolactone, p-dioxanone (1,4-dioxan-2-one), trimethylene carbonate (1,3-dioxan-2-one), δ-valerolactone, ε-decalactone, 2,5-diketomorpholine (morpholinedione), pivalactone, α,α-diethylpropiolactone, ethylene carbonate, ethylene oxalate, 3-methyl-1,4-dioxane-2,5-dione, 3,3-diethyl-1,4,dioxan-2,5-dione, γ-butyrolactone, 1,4-dioxepan-2-one, 1,5-dioxepan-2-one, 6,6-dimethyl-dioxepan-2-one, 6,8-dioxabicycloctane-7-one or combinations thereof. It is to be understood that the methods of the present invention can be applied to polymer blends.

Alternately the bioabsorbable polymers can be a component of a cross-linked network. That is, suitable polymers also include cross-linked polymers and hydrogels possessing hydrolysable ester or anhydride groups. It is to be understood that exemplary bioabsorbable, biocompatible polymers may be generally synthesized by a ring-opening polymerization of the corresponding lactone monomers or by polycondensation of the corresponding hydroxy-acids, or by combinations of these two polymerization methodologies.

As new absorbable polymers are being developed for medical devices and implants, a key issue is the length of time it will take for the material to disappear in the body, i.e., to absorb. Related to this issue is the desire to engineer medical devices and implants from bioabsorbable polymers that have desired absorption profiles in vivo. Although the definitive answer to this question is usually provided by preclinical studies using radiolabeled materials following the absorption, distribution, metabolism, and excretion of these materials and degradation products, other important means for studying bioabsorption include histology in which a measurement of the cross-sectional area of the implant is made as a function of time. For instance the paper entitled “Monocryl® Suture, a New Ultra-Pliable Absorbable Monofilament Suture”, by Rao S. Bezwada, Dennis D. Jamiolkowski, In-Young Lee, Vishvaroop Agarwal, Joseph Persivale, Susan Trenka-Benthin, Modesto Erneta, Jogendra Suryadevara, Alan Yang, and Sylvia Liu appearing in Biomaterials, Volume 16, Issue 15, October 1995, Pages 1141-1148 describes the biological performance of a monofilament suture based on caprolactone and glycolide. Another example of such studies is the work of Craig, P. H., Williams, J. A., Davis, K. W., Magoun, A. D., Levy, A. J., Bogdansky, S., and Jones, J. P. Jr. as reported in a paper entitled “A Biologic Comparison of Polyglactin 910 and Polyglycolic Acid Synthetic Absorbable Sutures” in Surg. Gynecol. Obstet., 141:1-10, 1975. Both of these papers are incorporated by reference.

Typically, in vivo performance of absorbable medical devices is commonly obtained in preclinical rat models. As described above, for sutures in vivo performance in Long-Evans rats has been used, where sutures are implanted in gluteal muscles and harvested at selected time points post-implantation where they are sectioned and stained for histological evaluation. In vivo absorption is thus typically evaluated in these models via tracking the disappearance of the implant in histologically prepared tissue sections.

The mechanical performance of absorbable medical devices changes with time in an in vivo environment. The failure mode of these devices may be dependent on one or more mechanical characteristics, for example, elongation-to-break, Young's Modulus, tensile strength, recovery characteristics, or tear strength. Since the mechanical performance is a function of the molecular weight and the molecular weight in turn depends on the extent of hydrolysis one might use the method of the present invention to predict mechanical property performance.

Although the hydrolysis profiler runs presented in the included examples hereinbelow were generally done at 75° C., other sufficiently effective temperature and pH conditions and other parameters can be utilized and explored, and correlations to in vivo behavior (such as absorption times or loss of mechanical properties) sought. A broad family of correlations may be possible provided that there are no major changes in the basic mechanisms of degradation. The temperature range may typically be greater than about 37° C., more typically about 60° C. to about 95° C., preferably about 70° C. to about 75° C., and most preferably about 70° C. The pH may range from typically greater than about 2 to about 11, more typically about 6.3 to about 8.3, and preferably about 7.3. The concentration of aqueous sodium hydroxide titrating base solution will typically be about 0.0001N to about 1.0N, more typically about 0.05N. The constant level of percent hydrolysis of the test article will typically be about 90% to about 100%, more typically about 95% to about 100%, preferably about 98% to about 100%, and even more preferably about 100%.

It is expected that changes in physical properties of a given material (such as suture breaking strength retention) are related to its hydrolysis profile as chemical degradation influences mechanical performance.

Since load bearing in semi-crystalline polymers is dependent on so-called “tie molecules” present in the amorphous phase but connecting crystallites, cleavage of these molecules, and not the chain segments in crystallites, controls strength retention. It is then expected that the amount of hydrolysis needed to occur to influence tensile strength in semi-crystalline polymers will be very small; within the first few percent after the hydrolysis of any residual monomer. To access this information experimentally, one may need to use a more dilute titrant, and/or a lower test temperature, and possibly increase the rate of data collection in the early part of the hydrolysis profile. A new set of correlation curves would need to be generated to relate the early portion of the hydrolysis profile to in vivo mechanical performance.

It is to be understood that high test temperatures might be limited by the boiling point of water. In cases where high acceleration is sought, a sealed system might be employed in which pressures greater than one atmosphere might be used.

It is also to be understood that relatively low test temperatures, provided that they are above body temperature, may be used. This may be particularly useful in the case of low-melting polymers. It is further understood that provided an activation energy of hydrolysis is known, data can be collected at a given test temperature and predictions of in vivo hydrolysis made using correlation curves based on in vitro data collected at a different temperature.

Regarding the role of sample size, it is to be understood that a sample size sufficiently large to effectively minimize experimental variation is required. When the sample size is too low variability in results will occur. It should be noted that very large sample sizes may then require very large hydrolysis reactors.

It is to be understood that a given correlation curve must be built using the same test conditions as is used for the test article being assessed.

Regarding the initial amount of water in the hydrolysis vessel, it is to be understood that a sufficient volume of water to effectively cover the test article in the hydrolysis vessel will be required. The hydrolysis vessel should have adequate spare volume to accommodate the test article, initial quantity of water and the final volume of titrating base solution.

It is to be understood that one could additionally include a color-changing pH indicator and a means of monitoring the color in order to control the titration to maintain the substantially constant test pH.

It should also be understood that in cases where enzymatic degradation pathways are significant, the in vitro to in vivo correlation may not hold. In these cases, one may need to add appropriate enzymes in suitable amounts to the reaction media.

A representative listing of bioabsorbable medical devices and implants that can be tested by the method of the present invention includes but is not limited to, for example, those devices presented in Tables 1 and 2, and equivalents.

TABLE 1
Filamentous Products Based on Synthetic Absorbable Polyesters

| | | | |
|---|---|---|---|
| Category | Company | Composition | Polymer Type |

Braided Sutures

| | | | |
|---|---|---|---|
| Coated VICRYL ™ | ETHICON, | L-Lactide and | Random |
| (polyglactin 910) Suture | Inc | Glycolide | Copolymer |
| | | | (dyed and |
| VICRYL RAPIDE ™ | ETHICON, | L-Lactide and | Random |
| (polyglactin 910) Suture | Inc | Glycolide | Copolymer |
| | | | (undyed) |
| Coated VICRYL ™ Plus | ETHICON, | L-Lactide and | Random |
| Antibacterial (polyglactin | Inc | Glycolide | Copolymer |
| 910) Suture | | | (dyed and |
| PANACRYL ™ Braided | ETHICON, | L-Lactide and | Random |
| Synthetic Absorbable | Inc | Glycolide | Copolymer |
| Suture | | | (undyed) |
| POLYSORB ™ Braided | Covidien | L-Lactide and | Copolymer |
| Suture | | Glycolide | (dyed and |
| | | | undyed) |
| DEXON ™ II Suture | Covidien | Glycolide | Homopolymer |
| | | | (dyed and |
| | | | undyed) |

Monofilament Sutures

| | | | |
|---|---|---|---|
| MONOCRYL ™ | ETHICON, | ε-Caprolactone | Segmented |
| (Poliglecaprone 25) | Inc | and Glycolide | Copolymer |
| Suture | | | (dyed and |
| | | | undyed) |
| PDS ™ II | ETHICON, | p-Dioxanone | Homopolymer |
| (polydioxanone) | Inc | | (dyed and |
| Suture | | | undyed) |
| CAPROSYN ™ Suture | Covidien | Glycolide, ε- | Tetrapolymer |
| | | Caprolactone, | (dyed and |
| | | Trimethylene | undyed) |
| | | Carbonate, and | |
| | | Lactide | |
| BIOSYN ™ Suture | Covidien | Glycolide, p- | Terpolymer |
| | | Dioxanone, and | (dyed and |
| | | Trimethylene | undyed) |
| | | Carbonate | |
| MAXON ™ Suture | Covidien | Glycolide and | Copolymer |
| | | Trimethylene | (dyed and |
| | | Carbonate | undyed) |

Meshes

| | | | |
|---|---|---|---|
| VICRYL ™ (polyglactin | ETHICON, | L-Lactide and | Random |
| 910) Knitted Mesh | Inc | Glycolide | Copolymer |
| VICRYL ™ (polyglactin | ETHICON, | L-Lactide and | Random |
| 910) Woven Mesh | Inc | Glycolide | Copolymer |

TABLE 2
Non-Filamentous Products Based on Synthetic Absorbable Polyesters

| | | | |
|---|---|---|---|
| Category | Company | Composition | Polymer Type |

Suture Anchors

| | | | |
|---|---|---|---|
| PANALOK ® RC Loop Anchor | DePuy | L-Lactide | Homopolymer |
| | Mitek | | |
| PANALOK ® Loop | DePuy | L-Lactide | Homopolymer |
| Anchor | Mitek | | |
| Bio-Statak ® Resorbable Soft | Zimmer | L-Lactide | Homopolymer |
| Tissue Attachment | | | |
| Device | | | |
| TAG ® Suture Anchors | Smith & | Trimethylene | Copolymer |
| (Wedge & Rod II Style) | Nephew | Carbonate and | |
| | | Glycolide | |
| TWINFIX AB ® | Smith & | L-Lactide | Homopolymer |
| 5.0 mm Suture Anchor | Nephew | | |
| OSTEORAPTOR ® | Smith & | PLLA-HA | Polylactic Acid |
| Anchor | Nephew | | Hydroxyapatite |
| LactoScrew ® Anchor | Biomet | L-Lactide and | Copolymer |
| | | Glycolide | |
| ALLthread ™ | Biomet | L-Lactide and | Copolymer |
| LactoSorb ® L15 Suture Anchors | | Glycolide | |
| BIOKNOTLESS ™ BR | DePuy | L-Lactide and | Composite of |
| Anchor | Mitek | Glycolide | Absorbable |
| | | Copolymer and | Copolymer and |
| | | β-TriCalcium | TCP |
| | | Phosphate | |
| | | (TCP) | |

Bone Pins

| | | | |
|---|---|---|---|
| OrthoSorb ® Resorbable | DePuy Ace | p-Dioxanone | Homopolymer |
| Tapered Pin | | | |
| SmartPin ® | formerly | SR-PLLA | Self Reinforced |
| | Bionx | | Homopolymer |
| | Implants | | |
| | Oy, now | | |
| | ConMed | | |
| | Livantec | | |
| | Biomaterials | | |
| RIGIDFIX ® ACL Cross | DePuy | L-Lactide | Homopolymer |
| Pin System | Mitek | | |

Plates

| | | | |
|---|---|---|---|
| MacroPore ™ Plate | Medtronic | L-Lactide and | Copolymer |
| | Neurosurgery | racemic D,L- | |
| | | Lactide | |
| Inion OTPS | Inion Ltd., | Polylactic acid/ | Copolymer |
| Biodegradable Mini | Tampere, | Trimethylene | |
| Plating System | Finland | Carbonate | |
| Resorb-X ® Plate | KLS Martin | Racemic D,L- | Copolymer (of |
| | | Lactide | L and D |
| | | | isomers) |

Ligating Clips

| | | | |
|---|---|---|---|
| ABSOLOK ™ Ligating Clip | ETHICON, | p-Dioxanone | Homopolymer |
| | Inc | | |

Suture Knot Clips

| | | | |
|---|---|---|---|
| LAPRA-TY ™ Suture | ETHICON, | p-Dioxanone | Homopolymer |
| Clip | Inc | | |

Tacks

| | | | |
|---|---|---|---|
| ArthroRivet ™ RC Tack | Biomet | L-Lactide and | Copolymer |
| | | Glycolide | |

Mesh Fixation Products

| | | | |
|---|---|---|---|
| SECURESTRAP ™ 5 mm | ETHICON, | Lactide, | Blend of |
| Absorbable Strap | Inc | Glycolide, and | random |
| Fixation Device | | p-Dioxanone | copolymer and |
| | | | homopolymer |
| AbsorbaTack ™ 5 mm | Covidien | Lactide and | Random |
| Fixation Device | | Glycolide | Copolymer |
| SorbaFix ™ Absorbable | Bard/Davol | L-Lactide and | Copolymer |
| Fixation System | | racemic D,L- | |
| | | Lactide | |

One way of viewing the hydrolysis of polyesters is as a “reverse polycondensation” process. One might then be able to utilize the mathematical relationships of polycondensation chemistry to gain insights into the degradation process. To do this it is necessary to state the definition of the term p, the so-called “extent of reaction”. In the present case, it can be thought of as the fraction of acid group moieties that exist as ester groups as opposed to free acid groups.

The extent of reaction, p, and the number average “degree of polymerization”, DPₙ, of a polyester of normal molecular weight distribution are related by the following equation:

DPₙ=1/(1−p)

The “degree of polymerization”, DP, is the number of repeat units in a chain; the corresponding value of DPₙ then refers to the entire population of chains. To achieve and maintain high mechanical properties, weight average molecular weight must be above a certain threshold.

An empirical relationship was found by Meng et al. [2] for an experimental multifilament suture polymer 90 mol % glycolide and 10 mol % lactide relating breaking strength retention (BSR) to molecular weight:

BSR=a+b ln M (5)

where M is either weight or number average molecular weight, having distinct “a” and “b” parameters, accordingly. For number average molecular weight, Mₙ, the parameters were found to be: a=446.17 and b=55.153 (the parameters were found to be independent of in vitro test temperature).

Solving equation 5, above, for M we obtain:

M=exp[(BSR−a)/b] (6)

The number average molecular weight is related to the molecular weight of a repeat unit and extent of reaction for a condensation polymer:

Mₙ=M₀/(1−p) (7)

Where M₀ is the molecular weight of the repeat unit and p is the extent of reaction.

For polyester degradation where “reverse polycondensation” is assumed, one may write

p=1−[COOH]/[COOH]_{∞} (8)

Where [COOH] is the concentration of carboxylic acid groups generated by hydrolysis of esters at any given time during hydrolytic degradation, and [COOH]_{∞} is the total amount of carboxylic acid groups to be generated at complete hydrolytic degradation (or the total amount of hydrolysable ester groups in the polymer).

Then one can express Mₙ as:

Mₙ=M₀/[COOH]/[COOH]_{∞} (9)

Substituting Mₙ into equation 6 above, we arrive at:

M₀/[COOH]/[COOH]_{∞}=exp[(BSR−a)/b] (10)

And solving for [COOH]/[COOH]_{∞} as BSR approaches zero we obtain

[COOH]/[COOH]_{∞}=M₀exp[a/b] (11)

Substituting in the values of M₀ and a and b gives

[COOH]/[COOH]_{∞}=1.82% (12)

Thus BSR is estimated to fall to zero at less than 2% of hydrolysis of the ester groups in the polymeric chain.

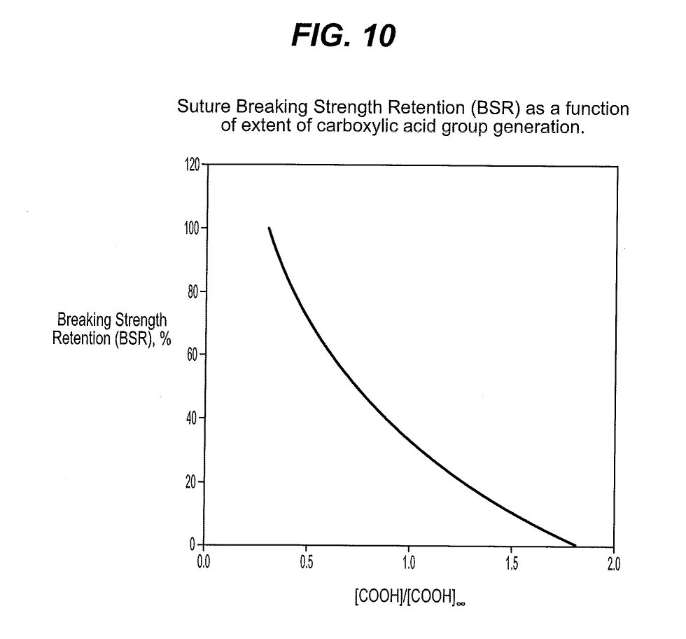

The empirically-derived relationship between BSR and extent of ester hydrolysis (via rearranging eq. 10) is plotted in FIG. 10.

It will be evident to one having ordinary skill to confirm the mapping of breaking strength retention to the extent of reaction.

To construct an in vivo-in vitro correlation curve (for instance of in vivo absorption time versus in vitro hydrolysis time) one might employ a variety of methods. It is useful to obtain a mathematical equation describing the relationship, whether it is linear or non-linear. If the response curve is linear, a well-accepted methodology of obtaining the mathematical descriptive equation is by performing a linear regression using the Method of Least Squares.

The novel in vitro method or methodology of the present invention, which is used to predict the in vivo absorption time of bioabsorbable polymeric implants and medical devices, has many advantages. The advantages include the following. It has been demonstrated that absorbable polyesters can be characterized for extent of hydrolytic degradation as a function of time under accelerated conditions. This includes above body temperature, and does not exclude temperatures above the glass transition temperature of the polymeric test article. Other means of acceleration of hydrolysis whereby degradation could occur at body temperature will be evident to one having ordinary skill, these include lower or higher pH than in the examples presented. Alternate means of acceleration may be useful when characterizing devices that may not be dimensionally stable (e.g., shrinking or melting) at elevated temperatures.

One of the utilities of the hydrolysis profiler technology is that it may reduce the need for animal testing. For example, to design in vivo tissue reaction and absorption studies on a new medical device based on a new absorbable polymer, it is necessary to conduct preclinical animal studies. For a new material, the end-point times for the preclinical studies are unknown, and additional animal groups are needed to ensure histology samples are collected during all significant material changes. The hydrolysis profiler may allow for the elimination of some of the extra animal groups, since the times of significant material change can be reasonably predicted.

Thus if one can reasonably predict that an absorbable polymeric medical device will absorb at approximately 180 days post implantation, one could focus on animal test periods centered in this time frame, rather than a larger number of more randomly selected test periods, which may not yield useful results. This then assists in establishing an effective animal testing plan.

In addition to decreasing the number of animals needed for testing, the improved efficiency gained from the methodologies of the present invention greatly reduce testing costs.

The following examples are illustrative of the principles and practice of the present invention although not limited thereto.

Commercially available suture products were tested as-received. Monomers were commercially available, polymerization-grade. Sodium hydroxide 0.05N was used as-received from Fisher Chemical (Fisher Scientific).

### Example 1

A pH-stat instrument: 718 STAT Titrator Complete, by MetroOhm, using Software TiNet 2.4 or later versions was employed. Samples were placed in a conventional 100 mL double-jacketed glass reaction vessel containing 75 mL of deionized water. The vessel was magnetically stirred, and was fitted with a sealed lid to prevent evaporation; a pressure of one atmosphere was maintained. The temperature of the stirred deionized water in the vessels was controlled to +/−0.1° C., and was maintained at a pH setpoint; a constant pH of 7.27 was used.

The sample vessel was continuously monitored for pH changes (drops in pH) from the setpoint. Typically the pH is controlled to ±0.2 or better. If any decrease was detected, 0.05N sodium hydroxide solution was added to return the pH to the setpoint. The pH, temperature, and volume of base, V(t), added to each hydrolysis vessel were recorded by computer as a function of time. Multiple setups were controlled by computer.

Prior to each sample run the pH probe at each test station was calibrated with pH 4.0, 7.0, and 10.0 standard solutions, calibrated at the test temperature. A typical sample size was 100 mg in 75 mL of deionized water, per test, titrated by 0.05N sodium hydroxide solution.

### Example 2

A variety of lactone monomers were used as model compounds in testing in accordance with example 1. Glycolide (1,4-dioxane-2,5-dione) was used to determine the reproducibility and accuracy of the method of the present invention.

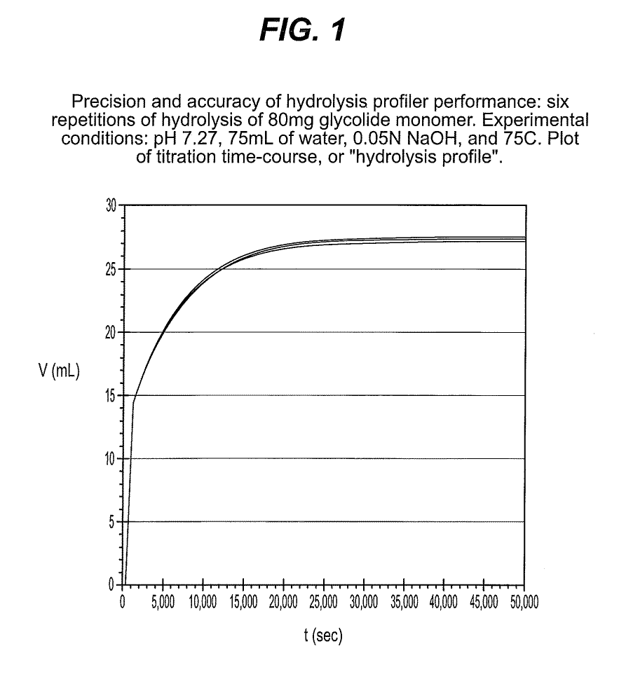

The hydrolysis profile can be expressed in a number of ways. Fundamentally, it is a measure of the extent of reaction of a test article with water as a function of time. FIG. 1 shows the time-course of titration as volume of added base with time, or “hydrolysis profile”. FIG. 1 shows hydrolysis profiles for glycolide monomer overlaid from six runs at 75° C. The reproducibility is good, as indicated by a 0.005 coefficient of variation (0.5% relative standard deviation) in the time necessary to achieve hydrolysis of 99% of the ester groups. The accuracy, determined by the deviation from the experimentally measured final volume (average of 27.3 mL) to the expected theoretical final volume (27.6 mL) has only a 1% disagreement.

The glycolide hydrolysis profile exhibits two features, an initial linear portion, followed by a curved portion. The initial linear portion corresponds to hydrolysis of one of the two carboxylic ester groups of the glycolide ring. This step is too fast to be tracked accurately by the system as configured. It should be clear that one could select more appropriate test conditions, for example lower the test temperature in order to collect accurate data for fast occurring events. Once the ring is cleaved, the now linear molecule, the carboxymethyl ester of hydroxyacetic acid (also known as glycolyl glycolate), contains one remaining ester; this ester exhibits a second, slower hydrolysis rate and is observed as the curved portion in the figure. Schematically the conversion of the lactone, glycolide, to two molecules of the hydroxy acid, glycolic acid, can be shown as:

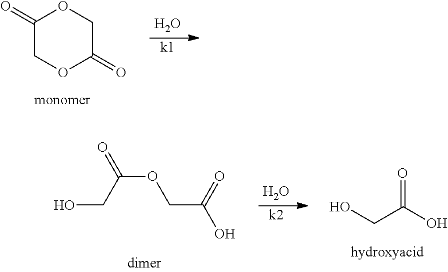

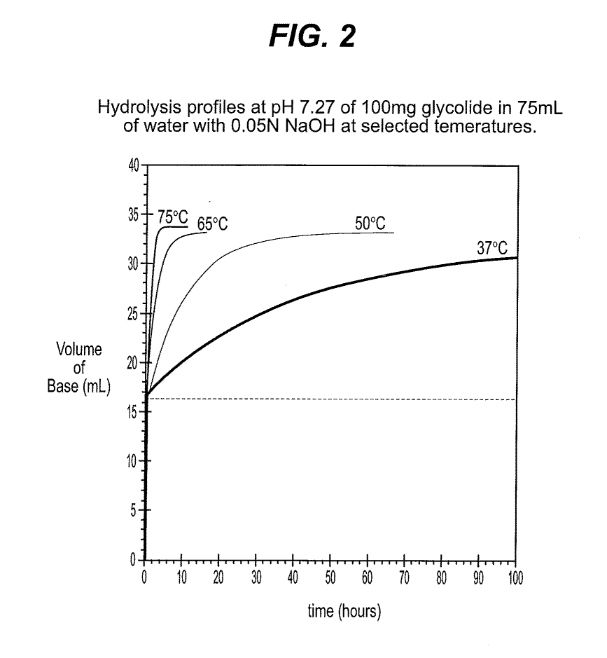

The kinetics of the reaction of the linear glycolic acid dimer, glycolyl glycolate, with water is temperature dependent, is as shown in FIG. 2.

Although we do not wish to be limited by scientific theory, the hydrolysis of the linear dimer into glycolic acid appears to be a first-order reaction. Since we are titrating with sodium hydroxide, a strong base, after the carboxylic acid group is formed by hydrolysis, it is immediately titrated and converted into the sodium salt. Thus, the volume of base added during the pH-stat titration, V(t), is proportional to the concentration of the sodium salt of the carboxylic acid groups, [COONa]. First-order kinetics relate the differential equation of change in sodium carboxylate groups with time to this instantaneous concentration:

$$\frac{d [ COONa ]}{d t} = {k}_{2} [ COONa ] \qquad ( 1 )$$

Integrating and substituting V(t) for [COONa], yields

V(t)=V_{∞}−(V_{∞}−V₁)e^{-k}^{₂}^{(t-t}^{₁}⁾ (2)

where the volume of base at the completion of hydrolysis from lactone monomer to linear dimer (at time t₁) is V₁, the final volume at very long times, when all ester groups have undergone hydrolysis, is V_{∞}, and the rate constant of conversion of the linear dimer to glycolic acid at a given reaction temperature is k₂.

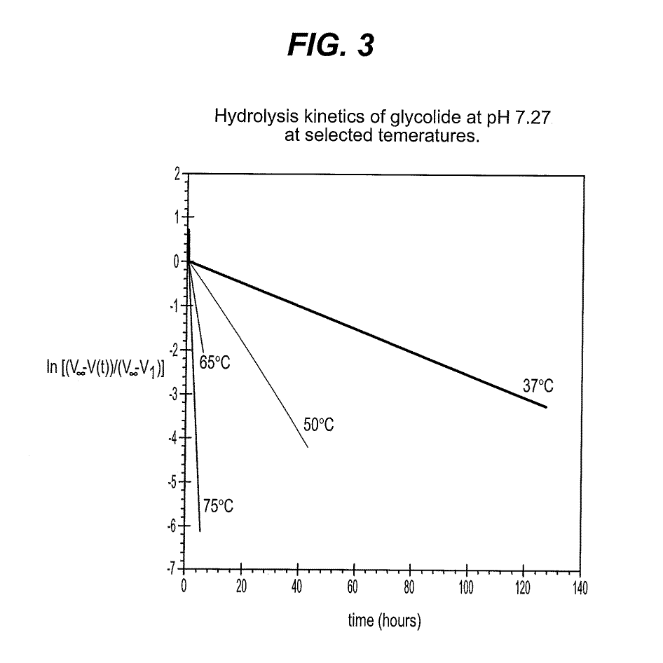

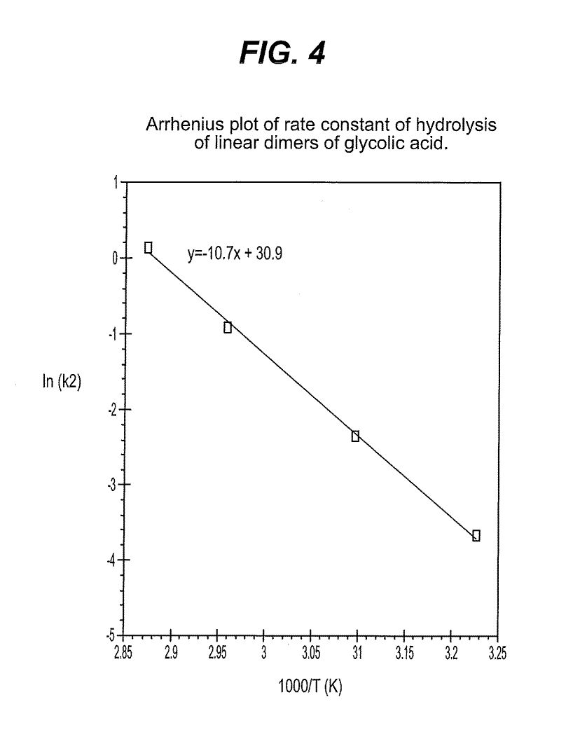

Equation 2 can be rearranged to allow for the computation of k₂ by linear regression, as is done with the data in FIG. 3. The slopes of the linear region in FIG. 3 yield the reaction constant, k₂ at each reaction temperature. A plot of the values of ln(k₂) for the hydrolysis of glycolyl glycolate vs. 1000/T are shown in FIG. 4. Arrhenius temperature dependence was observed:

$${k}_{2} = A {e}^{( \frac{- Ea}{RT} )} \qquad ( 3 )$$

where A is a constant (pre-exponential factor), Ea is the activation energy, R is the universal gas constant and T is the absolute temperature.

The activation energy for the hydrolysis of the linear glycolic acid dimer, glycolyl glycolate, was found to be 89.2 kJ/mol.

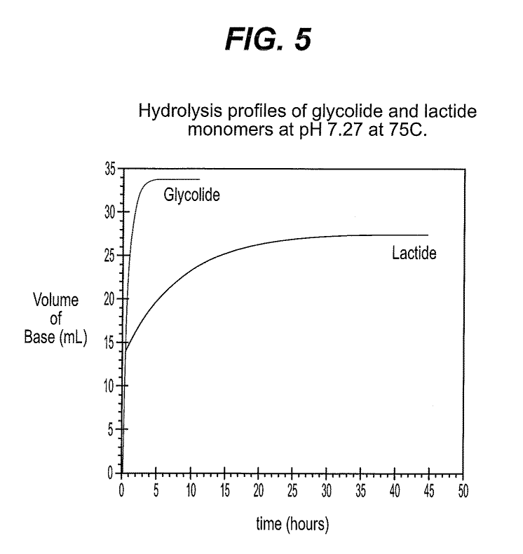

The early-time linear portion of FIG. 5 revealed that for both cyclic lactones, lactide and glycolide, at 75° C., there is essentially instantaneous (on the experimental time-scale) lactone ring-opening hydrolysis to the linear dimer form, with subsequent slower hydrolysis of these linear dimers into the corresponding hydroxy-acids. The rate constant k1, corresponding to ring-opening, is expected to be influenced by the ring-strain in the various lactones. At a given temperature, glycolide dimers were found to hydrolyze more rapidly than lactide dimers.

### Example 3

Having established the accuracy and experimental capability to conduct the hydrolytic degradation at temperatures as high as 75° C. in model compounds, more complex hydrolyzable polymeric materials were investigated next, such as those used to make absorbable sutures.

To determine whether an absorbable suture can be hydrolytically degraded at elevated temperatures without introducing physiologically irrelevant effects such as different chemical reactions, effects from surpassing the glass transition temperature (Tg) of the sample, changes in polymer morphology (e.g., crystallinity) or other changes that would not be found at body temperature, hydrolysis profiles on the following ETHICON brand sutures: Coated VICRYL™ (polyglactin 910) Suture and VICRYL RAPIDE™ (polyglactin 910) Suture were conducted at selected temperatures up to 75° C. (available from Ethicon, Inc., Somerville, N.J. 08876). This testing was conducted in accordance with the method of Example 1.

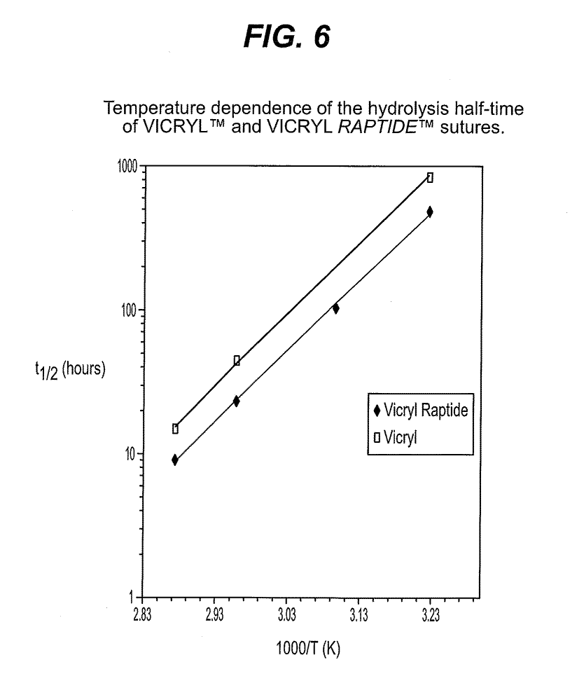

It should be noted that Reed and Gilding [16] and Agrawal et al. [17] suggest a dramatic transition in hydrolytic degradation kinetics at temperatures above Tg for PLGA polymers, and Buchanan et al. also raise concerns about elevated-temperature accelerated degradation testing at temperatures above Tg [18, 19]. However, the linear Arrhenius plot of the time needed to hydrolyze half of the ester groups in the polymer against 1/T in FIG. 6 in the present application does not bear this out. This is then supportive of the validity of our use of the chosen test temperature. That is, the fact that the Arrhenius plot of FIG. 6 is linear for a given suture suggests no change in reaction mechanism up to 75° C., and supports the rationale for correlating accelerated data at temperatures above the Tg of the bulk polymers to in vivo conditions. Note that the Tg of Coated VICRYL Suture is approximately 60° C., but when it is incubated in phosphate buffered saline at 37° C. for 24 hours the Tg decreases to approximately 30° C.[20]. The decrease in Tg of PLGA polymers during hydrolytic degradation is known [21, 22].

From the linear regression of the Arrhenius plot of FIG. 6 the activation energy for the hydrolysis of Coated VICRYL Suture was calculated to be 94.6 kJ/mol and the corresponding value for VICRYL RAPIDE Suture was 93.5 kJ/mol. These values are in reasonable agreement to literature values of PLGA polymers [17, 22].

A term is now introduced, tₓ, to designate the time necessary to hydrolyze x percent of the total hydrolyzable groups present. Thus t₅ refers to the time necessary for 5 percent of hydrolyzable groups to react, etc. It will be shown below that t₉₀ values for different absorbable polyesters can be correlated to in vivo absorption times. Additionally, it is believed that the time when mechanical failure of absorbable devices occurs may ultimately be correlated to their corresponding tₓ values when x is small (less than 5 percent). This is based on the fact that relatively few chains need to be cleaved to have mechanical failure. For purposes of illustration, in FIG. 6 we have selected t₅₀ (the time at which 50% of degradation has occurred) as a metric for the rate of degradation.

The level of hydrolysis that is appropriate for correlating the in vitro performance with the in vivo performance will be polymer-dependent. For a polymer having uniform monomer sequence distribution in which ester hydrolysis is random. It is possible for instance, to correlate the t₉₅ or the t₉₈ values to the in vivo absorption times. Other tₓ values can be correlated to in vivo absorption times.

It was found that much earlier t values could be correlated to the corresponding in vivo absorption times, and there would be an advantage to having a shorter testing time. Each parameter selection would only result in a different mathematical relation provided the basic degradation mechanisms were the same. It was found, however, that when the polyesters examined hydrolyzed to the point where 90 percent of the ester groups were hydrolyzed, the polyester test articles were water soluble at the elevated test temperature of 75° C. This test temperature was then selected for any additional work.

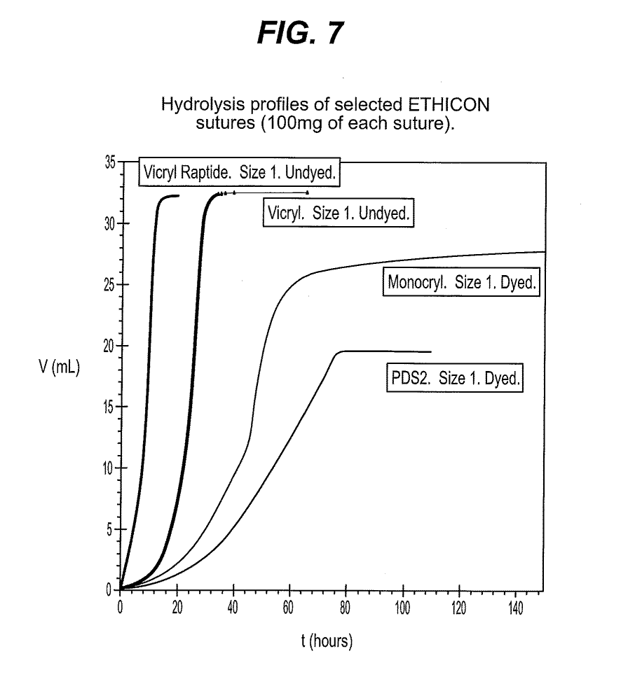

The hydrolysis profiles were collected at 75° C. of a variety of selected ETHICON brand sutures of a given size (size 1, 0.5 mm O.D.), available from Ethicon, Inc., and the results are shown in FIG. 7. These sutures range from the relatively long-lasting PDS™ II (polydioxanone) suture to the quickly absorbing VICRYL RAPIDE™ Suture. Coated VICRYL™ and VICRYL RAPIDE™ sutures are multifilament sutures, while MONOCRYL™ (poliglecaprone 25) and PDS II sutures are monofilament sutures, these last two sutures inherently have glass transition temperatures below room temperature. This figure also demonstrates the fact that since these sutures are made from different monomers the final volume of sodium hydroxide used to titrate the 100 mg samples will be different. The final volume depends on the amount of carboxylic acid groups generated per gram of sample; this relationship is presented below:

$$Vf = \frac{samplewt ( g )}{base ( mol / L )} [ \frac{mol \% C 1}{C 1 repeatMw ( g / mol )} + \frac{mol \% C 2}{C 2 repeatMw ( g / mol )} + \frac{mol \% Cn}{CnrepeatMw ( g / mol )} ] \qquad ( 4 )$$

Where V_{f} is final titration volume and Cₙ is mol % concentration of monomer n. Table 3, below, contains predicted and actual final titration volumes for 100 mg samples of selected absorbable polyesters.

TABLE 3
Predicted and Actual Final Titration Volumes for 100 mg Samples
of Selected Absorbable Polyester Sutures.

| | | Predicted | | |
|---|---|---|---|---|
| | | Titration | Actual | |
| ETHICON | Composition | Volume | Titration | % |
| Suture | (mol %) | (mL) | Volume (mL) | difference |
| VICRYL | 90% GLY, | 33.8 | 31.9 | 5.6 |
| RAPIDE ® | 10% LAC | | | |
| Suture | | | | |
| VICRYL ® | 90% GLY, | 33.8 | 32.2 | 4.7 |
| Suture | 10% LAC | | | |
| MONOCRYL ® | 75% GLY, | 29.3 | 27.5 | 6.2 |
| Suture | 25% CAP | | | |
| PDS II ® Suture | 100% PDO | 19.6 | 19.4 | 1.0 |

Where GLY is glycolide, LAC is lactide, CAP is ε-caprolactone, and PDO is p-dioxanone repeat units.

### Example 4

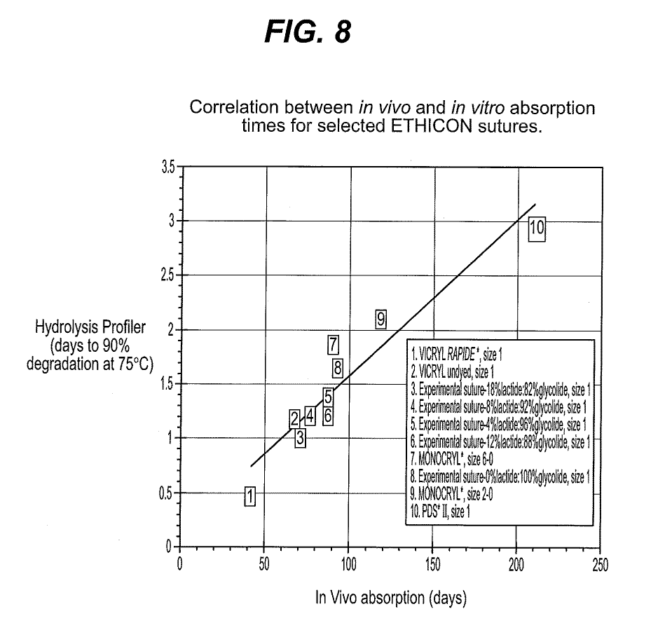

A plot of in vivo absorption time (via histology from intramuscular rat model studies) versus t₉₀ from a hydrolysis profile generated at 75° C. is shown in FIG. 8. This testing was done in accordance with the method of Example 1. Again, t₉₀ is defined as the point in the time-course where 90% of available ester groups have hydrolyzed. The selection of the time of 90% ester hydrolysis was made on the basis of experimental convenience and relevance to in vivo end-points. A linear regression gives the relationship: y=0.014x+0.137 with an R2 value of 0.904. The regression correlation coefficient, R2, of 0.904 indicates a good correlation between the in vitro t₉₀ value and the in vivo absorption time.

The methods described in the above examples allow the prediction of the in vivo absorption time of a test sample in the following way. One first generates a correlation curve of in vivo absorption time vs in vitro hydrolysis time generated at a given test temperature, set pH condition and extent of hydrolysis (tₓ) value. One then generates under similar in vitro test conditions the value of in vitro hydrolysis time. Utilizing this in vitro hydrolysis time and the correlation curve one can predict the in vivo absorption time of the test article.

### Example 5

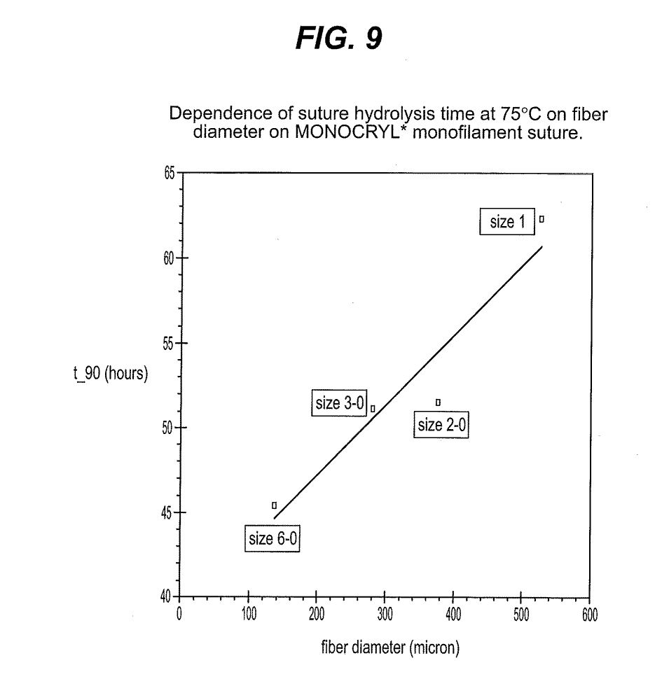

There are many factors controlling hydrolytic degradation. One is the surface area of the absorbable device. For monofilament sutures, such as MONOCRYL™ suture, the degradation time is related to filament diameter. It is not unexpected to find that larger diameter monofilament sutures require longer times for the diffusion of water into the filament interior, leading to longer degradation times; this relationship is presented in FIG. 9 where t90 is plotted against MONOCRYL™ Suture fiber diameter, using the method of Example 1.

### Example 6

The multifilament braided suture commercially available and known as Vicryl™ 2-0 Sutre was subjected to the testing using the method of the present invention, in accordance with Example 1. Testing temperatures included 50° C., 60° C., 70° C. and 80° C. to generate hydrolysis profiles. With regard to analysis of the generated curves, the times necessary to achieve an extent of hydrolysis of 10, 50, 90, and 98 percent hydrolysis were recorded for each of the test articles tested at each temperature.

TABLE 4
Degradation time of Vicryl ® 2-0 Sutures at Various Temperatures

| Temperature | 10% | 50% | 90% | 98% |
|---|---|---|---|---|
| (Vicryl ® | Degradation | Degradation | Degradation | Degradation |
| Suture) | (Hours) | (Hours) | (Hours) | (Hours) |
| 50° C. | 127 | 206 | 265 | 302 |
| 60° C. | 49 | 87 | 112 | 130 |
| 70° C. | 14 | 26 | 33 | 37 |
| 80° C. | 8 | 14 | 18 | 20 |

The inverse of the time for degradation (as measured in seconds) was plotted against inverse temperature (in Kelvin). The Arrhenius values were calculated from the equation of the line. The activation energy at 10% degradation, 50% degradation, 90% degradation and 98% degradation was calculated from the slope of their respective equations.

TABLE 5
Activation Energy Values Calculated from the Arrhenius Plots

| | 10% | 50% | 90% | 98% |
|---|---|---|---|---|
| | Degradation | Degradation | Degradation | Degradation |
| | Ea | Ea | Ea | Ea |
| Sutures | (K J mol⁻¹) | (K J mol⁻¹) | (K J mol⁻¹) | (K J mol⁻¹) |
| Vicryl | 89 | 81 | 81 | 81 |
| Rapide ® 3-0 | | | | |
| Vicryl ® 2-0 | 91 | 88 | 89 | 89 |
| Monocryl ® | 76 | 79 | 80 | 80 |
| 2-0 | | | | |
| PDS II ® 2-0 | 119 | 106 | 104 | 103 |

From the calculated Arrhenius values the degradation time of the sutures at body temperature (37° C.) was determined.

TABLE 6
Predicted Degradation of the Sutures at 37 Degree Centigrade
using Arrhenius Equation

| | 10% | | | |
|---|---|---|---|---|
| | De- | 50% | 90% | 98% |
| | gradation | Degradation | Degradation | Degradation |
| | (Hours) | (Hours) | (Hours) | (Hours) |
| Vicryl Rapide ® | 8 | 18 | 26 | 31 |
| 3-0 Suture | | | | |
| Vicryl ® 2-0 | 21 | 34 | 44 | 52 |
| Suture | | | | |
| Monocryl ® 2-0 | 20 | 45 | 65 | 79 |
| Suture | | | | |
| PDS II ® 2-0 | 246 | 278 | 311 | 321 |
| Suture | | | | |

The four linear curves corresponding to an extent of hydrolysis 10, 50, 90 and 98% had correlation coefficients greater than 0.985. The Arrhenius plot correlation coefficient for this wide variety of absorbable polymers indicate strong linearity across the temperature range from 50° to 80° C. These test temperatures are above the glass transition temperatures of the sutures tested.

### Example 7

The multifilament braided suture commercially available and known as Vicryl Rapide™ 3-0 suture was subjected to the testing using the method of the present invention, in accordance with Example 1. Testing temperatures included 50° C., 60° C., 70° C. and 80° C. to generate hydrolysis profiles. With regards to analysis of the generated curves, the times necessary to achieve an extent of hydrolysis of 50, 90, and 98 percent hydrolysis were recorded for each of the test articles tested at each temperature.

TABLE 7
Degradation time of Vicryl Rapide 3-0 Sutures at Various
Temperatures

| Temperature | | | | |
|---|---|---|---|---|
| (Vicryl | 10% | 50% | 90% | 98% |
| Rapide ™ | Degradation | Degradation | Degradation | Degradation |
| Suture) | (Hours) | (Hours) | (Hours) | (Hours) |
| 50° C. | 52 | 119 | 172 | 204 |
| 60° C. | 23 | 49 | 70 | 88 |
| 70° C. | 11 | 22 | 30 | 35 |
| 80° C. | 4 | 9 | 13 | 16 |

The inverse of the time for degradation (as measured in seconds) was plotted against inverse temperature (in Kelvin). The Arrhenius values were calculated from the equation of the line. The activation energy at 10% degradation, 50% degradation, 90% degradation and 98% degradation was calculated from the slope of their respective equations.

For Vicryl Rapide™ 3-0 Suture, the four linear curves corresponding to an extent of hydrolysis 10, 50, 90 and 98% had correlation coefficients greater than 0.992. The Arrhenius plot correlation coefficient indicates strong linearity across the temperature range from 50° to 80° C. These test temperatures are above the glass transition temperatures of the sutures tested.

### Example 8

The monofilament suture commercially available and known as Monocryl™ 2-0 suture was subjected to the testing using the method of the present invention, in accordance with Example 1. Testing temperatures included 50° C., 60° C., 70° C. and 80° C. to generate hydrolysis profiles. With regard to analysis of the generated curves, the times necessary to achieve an extent of hydrolysis of 50, 90, and 98 percent hydrolysis were recorded for each of the test articles tested at each temperature.

TABLE 8
Degradation time of Monocryl Sutures at Various Temperatures

| | 10% | | | |
|---|---|---|---|---|
| | De- | 50% | 90% | 98% |
| Temperature | gradation | Degradation | Degradation | Degradation |
| (MonocrylSuture) | (Hours) | (Hours) | (Hours) | (Hours) |
| 50° C. | 138 | 303 | 420 | 498 |
| 60° C. | 68 | 144 | 212 | 268 |
| 70° C. | 27 | 56 | 75 | 85 |
| 80° C. | 13 | 26 | 36 | 44 |

For Monocryl™ 2-0 suture, the four linear curves corresponding to an extent of hydrolysis 10, 50, 90 and 98% had correlation coefficients greater than 0.984. The Arrhenius plot correlation coefficient indicates strong linearity across the temperature range from 50° to 80° C. These test temperatures are above the glass transition temperatures of the sutures tested.

### Example 9

The monofilament suture commercially available and known as PDSII 2-0 suture was subjected to testing using the method of the present invention, in accordance with Example 1. Testing temperatures included 50° C., 60° C., 70° C. and 80° C. to generate hydrolysis profiles. With regard to analysis of the generated curves, the times necessary to achieve an extent of hydrolysis of 10, 50, 90, and 98 percent hydrolysis were recorded for each of the test articles tested at each temperature.

TABLE 9
Degradation time of PDS II 2-0 Sutures at Various Temperatures

| | 10% | 50% | 90% | 98% |
|---|---|---|---|---|
| Temperature | degradation | degradation | degradation | degradation |
| (PDS II) | (Hours) | (Hours) | (Hours) | (Hours) |
| 60° C. | 241 | 395 | 469 | 501 |
| 70° C. | 69 | 121 | 149 | 157 |
| 80° C. | 21 | 45 | 56 | 61 |

For PDSII™ 2-0 suture, the four linear curves corresponding to an extent of hydrolysis 10, 50, 90 and 98% had correlation coefficients greater than 0.998. These Arrhenius plot correlation coefficient indicates strong linearity across the temperature range from 50° to 80° C. These test temperatures are above the glass transition temperatures of the sutures tested.

Although this invention has been shown and described with respect to detailed embodiments thereof, it will be understood by those skilled in the art that various changes in form and detail thereof may be made without departing from the spirit and scope of the claimed invention.

### REFERENCES

[1] Ceonzo K, Gaynor A, Shaffer L, Kojima K, Vacanti C A, Stahl G L. Polyglycolic Acid-Induced Inflammation: Role of Hydrolysis and Resulting Complement Activation. Tissue Eng 2006; 12(2):301-8.
[2] Deng M, Zhou J, Chen G, Burkley D, Xu Y, Jamiolkowski D, Barbolt T. Effect of load and temperature on in vitro degradation of poly(glycolide-co-L-lactide) multifilament braids. Biomaterials 2005; 26:4327-4336
[3] Sawhney A S, Hubbell J A. Rapidly degraded terpolymers of dl-lactide, glycolide, and ε-caprolactone with increased hydrophilicity by copolymerization with polyethers. J Biomed Mat Res 1990; 24:1397-1411.
[4] Johansson H, Sebelius H. Saponification of glycolide and lactide in acid solution. Berichte der Deutschen Chemischen Gesellschaft [Abteilung] B: Abhandlungen 1919, 52B 745-52.
[5] Ringer O, Skrabal A. Hydrolysis of lactic acid lactide. Monatshefte fuer Chemie 1923; 43:507-23.
[6] Mhala M M, Mishra J P. Hydrolysis of dl-lactide. Indian Journal of Chemistry 1970; 8(3):243-6.
[7] Mhala M M, Mishra J P, Ingle T S. Kinetics of hydrolysis of glycolide. Indian Journal of Chemistry 1972; 10(10):1006-10.
[8] Lanza P. Multi-purpose recording pH meter. Journal of Electroanalytical Chemistry 1967; 13(1-2):67-72.
[9] Salmi E J, Leino E. The alkaline hydrolysis of esters of glycolic, lactic, and a-hydroxyisobutyric acids. Suomen Kemistilehti B 1944; 17B:19-21.
[10] Tsibanov V V, Loginova T A, Neklyudov A D. Analysis of pH-stat curves of enzymic hydrolysis in variable volumes of solution. Zhurnal Fizicheskoi Khimii 1982; 56(5): 1183-8.
[11] Williams K R. Automatic titrators in the analytical and physical chemistry laboratories. Journal of Chemical Education 1998; 75(9):1133-1134
[12] Baran J, Penczek S. Hydrolysis of Polyesters of Phosphoric Acid. 1. Kinetics and the pH Profile. Macromolecules 1995; 28(15):5167-76.
[13] Tunc D C, Goekbora M, Higham P. A new method for the estimation for the absorption time of bioabsorbable polymers in the body. Technology and health care: official journal of the European Society for Engineering and Medicine 2002; 10(3-4):237-42.
[14] Soucek M D, Johnson A H. New intramolecular effect observed for polyesters: An anomeric effect. JCT Research 2004; 1(2):111-116.
[15] Gebauer B, Jendrossek D. Assay of poly(3-hydroxybutyrate) depolymerase activity and product determination. Applied and Environmental Microbiology 2006; 72(9):6094-6100.
[16] Gilding D K, Reed A M. Biodegradable polymers for use in surgery-poly(glycolic)/poly(lactic acid) homo and copolymers: 2. in vitro degradation. Polymer 1981; 22:494-8.
[17] Agrawal C M, Huang D, Schmitz J P, Athanasiou K A. Elevated Temperature Degradation of a 50:50 Copolymer of PLA-PGA. Tissue Engineering 1997; 3(4):345-352.
[18] Weir N A, Buchanan F J, On J F, Farrar D F, Dickson G R. Degradation of poly-L-lactide. Part 2: increased temperature accelerated degradation. Proc Inst Mech Eng 2004; 218(5):321-30.
[19] Degradation rate of bioresorbable materials. Edited by Fraser Buchanan. Woodhead Pulblishing Limited, 2008.
[20] Ethicon Data on File
[21] Shah S, Cha Y and Pitt C G. Poly(glycolic acid-do-DL-lactic acid): Diffusion or degradation controlled drug delivery? J. Controlled Release 1992; 18:261-270.
[22] Dunne M, Corrigan (O), Ramtoola Z. Influence of particle size and dissolution conditions on the degradation properties of polylactide-co-glycolide particles. Biomaterials 2000; 21(16):1659-1668.

We claim:

1. A method of predicting the in vivo behavior of synthetic absorbable polymers, their implants or medical devices formed therefrom, possessing hydrolysable linkages within the chain, based on an in vitro test, comprising the steps of:
(a) subjecting a known quantity of a test article of known in vivo absorption time to hydrolysis at a substantially constant pH and at a substantially constant test temperature above or at body temperature using a known concentration of titrating base, recording the volume of titrating base with time;
(b) recording the time necessary to achieve a constant level of percent hydrolysis of the test article wherein said percent hydrolysis is 70 percent or greater;
(c) repeating steps (a) and (b) utilizing the test conditions selected for steps (a) and (b) with at least one different test article of different known in vivo absorption times;
(d) constructing an in vivo-in vitro correlation curve of in vivo absorption time versus in vitro hydrolysis time as recorded in step (b);
(e) subjecting a known quantity of test article of unknown in vivo absorption time to hydrolysis at the test conditions selected for steps (a) and (b) using a known concentration of titrating base, recording the volume of titrating base with time;
(f) predicting the in vivo behavior utilizing the correlation curve of step (d) and the in vitro hydrolysis time of step (e).

2. The method of claim 1, wherein said test temperature is within the range of greater than about 60° C. to about 95° C.

3. The method of claim 1, wherein said test temperature is within the range of about 70° C. to about 75° C.

4. The method of claim 1, wherein said test temperature is about 70° C.

5. The method of claim 1, wherein said constant pH is within the range of about 2 to about 11.

6. The method of claim 1, wherein said constant pH is within the range of about 6.3 to about 8.3.

7. The method of claim 1, wherein said constant pH is 7.3.

8. The method of claim 1, wherein said titrating base is an aqueous sodium hydroxide solution.

9. The method of claim 8, wherein said aqueous sodium hydroxide solution has a concentration within the range of about 0.0001N to about 1.0N.

10. The method of claim 8, wherein said aqueous sodium hydroxide solution has a concentration of about 0.05N.

11. The method of claim 1, wherein said test article of unknown in vivo absorption time is in the form of a monofilament.

12. The method of claim 1, wherein said test article of unknown in vivo absorption time is in the form of a multifilament.

13. The method of claim 1, wherein said test article of unknown in vivo absorption time is in the form of a non-filamentous implantable medical device.

14. The method of claim 1, additionally including a color-changing pH indicator and a means of monitoring the color in order to control the titration to maintain said substantially constant pH.

15. The method of claim 1, wherein the said constant level of percent hydrolysis of the test article is within the range of about 90% to about 100%.

16. The method of claim 1, wherein the said constant level of percent hydrolysis of the test article is within the range of about 95% to about 100%.

17. The method of claim 1, wherein the said constant level of percent hydrolysis of the test article is within the range of about 98% to about 100%.

18. The method of claim 1, wherein the said constant level of percent hydrolysis of the test article is about 100%.

19. The method of claim 1, wherein the synthetic absorbable polymer their implants or medical devices formed therefrom is selected from the group consisting of aliphatic polyesters, poly(amino acids), copoly(ether-esters), polyalkylene oxalates, polyalkylene diglycolates, polyamides, tyrosine-derived polycarbonates, poly(iminocarbonates), polyorthoesters, polyoxaesters, polyamidoesters, polyoxaesters containing amine groups, poly(anhydrides), polyphosphazenes, polypropylene fumarates), absorbable poly(ester urethanes), and combinations and blends thereof.

20. A method of predicting the in vivo absorption time of synthetic absorbable polymers, their implants or medical devices formed therefrom, possessing hydrolysable linkages within the chain, based on an in vitro test, comprising the steps of:
(a) subjecting a known quantity of a test article of known in vivo absorption time to hydrolysis at a substantially constant pH and at a substantially constant test temperature above or at body temperature using a known concentration of titrating base, recording the volume of titrating base with time;
(b) recording the time necessary to achieve a constant level of percent hydrolysis of the test article wherein said percent hydrolysis is 70 percent or greater;
(c) constructing an in vivo-in vitro correlation curve of in vivo absorption time versus in vitro hydrolysis time as recorded in step (b);
(d) subjecting a known quantity of test article of unknown in vivo absorption time to hydrolysis at the test conditions selected for steps (a) and (b) using a known concentration of titrating base, recording the volume of titrating base with time;
(e) predicting the in vivo absorption time utilizing the correlation curve of step (c) and the in vitro hydrolysis time of step (d).

21. The method of claim 20, wherein said test temperature is within the range of about 60° C. to about 95° C.

22. The method of claim 20, wherein, said test temperature is within the range of about 70° C. to about 75° C.

23. The method of claim 20, wherein said test temperature is about 70° C.

24. The method of claim 20, wherein said constant pH is within the range of about 2 to about 11.

25. The method of claim 20, wherein said constant pH is within the range of about 6.3 to about 8.3.

26. The method of claim 20, wherein said constant pH is about 7.3.

27. The method of claim 20, wherein said titrating base is an aqueous sodium hydroxide solution.

28. The method of claim 27, wherein said aqueous sodium hydroxide solution has a concentration within the range of about 0.0001N to about 1.0N.

29. The method of claim 27, wherein said aqueous sodium hydroxide solution has a concentration of about 0.05N.

30. The method of claim 20, wherein said test article of unknown in vivo absorption time is in the form of a monofilament.

31. The method of claim 20, wherein said test article of unknown in vivo absorption time is in the form of a multifilament.

32. The method of claim 20, wherein said test article of unknown in vivo absorption time is in the form of a non-filamentous implantable medical device.

33. The method of claim 20, additionally including a color-changing pH indicator and a means of monitoring the color in order to control the titration to maintain said substantially constant pH.

34. The method of claim 20, wherein the said constant level of percent hydrolysis of the test article is within the range of about 90% to about 100%.

35. The method of claim 20, wherein the said constant level of percent hydrolysis of the test article is within the range of about 95% to about 100%.

36. The method of claim 20, wherein the said constant level of percent hydrolysis of the test article is within the range of about 98% to about 100%.

37. The method of claim 20, wherein the said constant level of percent hydrolysis of the test article is about 100%.

38. The method of claim 20, wherein the synthetic absorbable polymer their implants or medical devices formed therefrom is selected from the group consisting of aliphatic polyesters, poly(amino acids), copoly(ether-esters), polyalkylene oxalates, polyalkylene diglycolates, polyamides, tyrosine-derived polycarbonates, poly(iminocarbonates), polyorthoesters, polyoxaesters, polyamidoesters, polyoxaesters containing amine groups, poly(anhydrides), polyphosphazenes, polypropylene fumarates), absorbable poly(ester urethanes), and combinations and blends thereof.

39. The method of claim 1, wherein the substantially constant test temperature is greater than about 37° C.

40. The method of claim 20 wherein the substantially constant test temperature is greater than about 37° C.
